(19) United States
(12) Patent Application Publication
(10) Pub. No.: US 2023/0330297 A1
(43) Pub. Date: Oct. 19, 2023

(54) HOMEOSTATIC SINGLE LAYER DRESSING

(71) Applicant: Waldir Teixeira RENÓ, Guaratingueta (BR)

(72) Inventor: Waldir Teixeira RENÓ, Guaratingueta (BR)

(21) Appl. No.: 17/721,853

(22) Filed: Apr. 15, 2022

(51) Int. Cl. C08L 23/06; A61L 15/42; A61L 15/58; A61L 15/24

(52) U.S. Cl. CPC A61L 15/24; A61L 15/425; A61L 15/58; C08L 23/06; A61L 2400/04

(57) ABSTRACT

A dressing capable of treating wounds of various origins through the homeostatic activity it exerts on the injured tissue. Thus, it regulates the dilation of the vessels belonging to the microcirculation, allowing greater control in the production of fluids and secretions, as well as limiting their presence in the wound bed by a pressure gradient that sweeps the secretions to the periphery of the dressing, favoring the process of tissue regeneration and healing.

### FIELD OF INVENTION

The present invention relates to the field of medical dressing technology, specifically provides a single layer dressing for the care and treatment of acute and chronic wounds, surgical wounds, and trauma injuries.

In particular, the present invention reveals a single layer dressing which exerts by itself a homeostatic activity on the injured tissue and, thus, its light pressure regulates the dilation of the vessels belonging to the microcirculation and control the neurotransmission, and its molecular structure regulates the hydraulic gradient and thermal function. In this sense, this dressing controls the production of fluids and secretions in the wound and does not allow the accumulation of these substances in the wound bed, favoring the processes of tissue regeneration and healing.

### BACKGROUND

The skin is the largest organ in the human body and acts as a protective barrier between the outside and the inside of the body. The skin contains nerve endings that transmit sensations such as pain, pressure, touch, itchiness and temperature to the brain. Blood vessels found in the skin nourish it and control body temperature. Lymphatic vessels help ward off infections and other harmful substances.

As an organ responsible for protecting the body from external aggressions, the treatment of skin lesions, surgical or other traumatic causes, must be careful in order to restore the natural defense barrier and prevent microorganisms from falling into the bloodstream and causing damage to health. The treatment of skin lesions can be done surgically, or clinically with the use of dressings.

The choice of dressing takes into consideration the type of wound to be treated, whether deep or superficial, whether infected, whether necrotic and whether it is associated with diseases that hinder tissue repair. Dressings can consist of different materials and can be differentiated, according the stage of treatment of the wound in which the patient is.

The document US10472450, entitled “Acute care cover for severe injuries” reveals a polymeric mesh that, among other things, uses polyethylene glycol acrylate for the care and protection of wounds.

Documents US10123912 and US10123909, entitled, respectively, “Foldable absorbent apparatus and method of using the same” and “Wound dressing” describe devices used in the treatment of wounds that absorb the exudate produced by the injury.

Currently, pressure is used to treat wounds through the Negative Pressure Therapy strategy, which promotes healing in a humid environment, as the name says, by applying subatmospheric pressure (negative) in a controlled and local manner. The therapy comprises an interface material, which can be gauze or foam, applied on the wound bed, in direct contact with it, to cover its extension. Said material is then covered by an adhesive film, which completely occludes the wound and leaves it isolated from the external environment. Finally, a suction tube is connected to the system applied to the patient’s body and to the secretion reservoir, which is connected to a computerized device, to provide negative pressure to the wound bed and, thus, remove the exudate.

The foam used as the interface material is polyurethane, which has great elasticity, but which has the disadvantage of the fact that the tissue in formation can grow towards the pores of the foam and thus become entangled in their chains, which favors the occurrence of trauma and pain when changing the dressing.

The document US10406036 reveals a useful equipment for the application of Negative Pressure Therapy.

In addition to the potential adherence of the polyurethane dressing to the wound bed, caused both by the growth of the tissue in the direction of the foam, and by the application of pressure, causing trauma and hindering the regeneration of the skin. Negative Pressure Therapy also has other disadvantages, such as not being able to be used in tissues with malignancy, difficulty in application in patients who need an external fixator, contraindication for use in patients under anticoagulation therapy and contraindication in cases of excessively exudative wounds.

A final disadvantage in the application of current dressings is the generation of inadequate and unstable pressure on the wound which increases with the absorption of exudate and causes swelling of the injured tissue, a drop in temperature and inadequate blood flow, generating ischemia in the wound bed, still producing more secretions with exacerbation of exudate, forming a vicious cycle that promotes an environment conducive to the proliferation of microorganisms and biofilm formation.

In this way, it becomes clear the difference between the present invention and the solutions available in the state of the art, which demonstrates compliance with the requirements of novelty and inventive step of the material disclosed here.

### SUMMARY OF DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In order to overcome the disadvantages of currently available dressings, the present invention discloses a single layer dressing for treating wounds by homeostatic activity, which does not require the use of electronic devices and external forces to remove exudates from the traumatized area, in addition to carrying out the exchange of the dressing without pain, as the material does not adhere to the wound bed and, therefore, psychologically spares the patient and does not impair healing.

The dressing of the present invention does not require chemical additives and overlapping layers of different materials, thus reducing the dressing weight on the wound, and the incidence of allergies and potential chemical injury to the already traumatized tissue.

The dressing of the present invention also increases the local temperature, which stimulates the microcirculation of the injured tissue while its homeostatic pressure controls the vascular caliber, preventing inadequate vasodilation.

The dressing of the present invention has low elasticity and high flexibility, which allows changing the shape of the material and adapting it to any region of the body to be treated. Another advantage of changing the shape is the correction of the pressure gradient between the dressing center and the periphery through peristaltic movements.

Still further, the present invention discloses a method for applying the single layer dressing to an exposed wound, said dressing being capable of promoting homeostatic activity in the affected area, and, thus, promoting the best regeneration of the injured tissue.

The material used and the preparation of the dressing of the present invention allows its application in general surgeries, plastic surgeries, head and neck surgeries, skin cancer removal surgeries, burn treatments mainly in grafted areas promoting the integrity of the grafts, pressure ulcers, neoplasic skin lesions, and treatment of hypertrophic scars.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates exemplary homeostatic single layer dressings in various non-limiting formats (A, B, C, D, F) before being applied to the skin surface.

FIG. 2 illustrates the homeostatic single layer dressing format A and its application to the limb after severe trauma, demonstrating that the dressing does not adhere to the graft bed;

FIG. 3 illustrates the homeostatic single layer dressing format B applied to an extensive body surface after a second-degree burn, reducing fluid loss and pain and accelerating recovery.

FIG. 4 illustrates the homeostatic single layer dressing of format B applied to the burned fingers on the date of the accident and right after the skin graft with full integration followed by rapid recovery and without loss of mobility.

FIG. 5 illustrates the homeostatic dressing format C applied at the end of a breast surgery, observing that after removal of the dressing the apparatus maintains the anatomical shape of the applied site.

FIG. 6 is a close-up view of the homeostatic single layer dressings formats D and F and their applications at the end of a face and neck plastic surgery resulting in rapid recovery and reduction of edema and ecchymosis.

FIG. 7 is a close-up view of the homeostatic single layer dressing format E and its application at the end of an eyelid surgery with better control of bleeding and discomfort.

FIG. 8 is a close-up view of homeostatic single layer dressing format E and its removal after 5 days, revealing an operated area with minimal bruising and swelling and a dry dressing in contact with the wound.

FIG. 9 is a detailed view of the use of the homeostatic single layer dressing format F applied to fix a skin graft after cutaneous tumor excision, and dressing removal on the 4th day postoperative without adhesion and with dry inner surface with minimal secretion at the edge line.

FIG. 10 is a close-up view of the homeostatic single layer dressing format D applied at the end of a cervical surgery, specifically a thyroidectomy. Its removal after 3 days shows a dry and clean core and the presence of secretion only at the edge line, which demonstrates the homeostatic activity generating a hydraulic gradient that prevents the formation of a biological film and drastically reduces ecchymosis and edema.

FIG. 11 represents intraoperative and postoperative application of the homeostatic single layer dressing format F in a complex neck surgery, resulting in a pain-free and uncomplicated postoperative period.

FIG. 12 illustrates the application of the homeostatic single layer dressing format F as a long plate for use postoperative under the garment to control edema, ecchymosis and pain;

FIG. 13 demonstrates the application of the homeostatic single layer dressings formats E and F under pressure garment for the treatment of hypertrophic scar, resulting in relief of discomfort and pain, and regression of hypertrophy

FIG. 14 demonstrates the format F with the inner surface filled with ointment and applied to an infected ulcer, resulting in rapid recovery and demonstrating the drug-carrying activity of the dressing.

### DETAILED DESCRIPTION

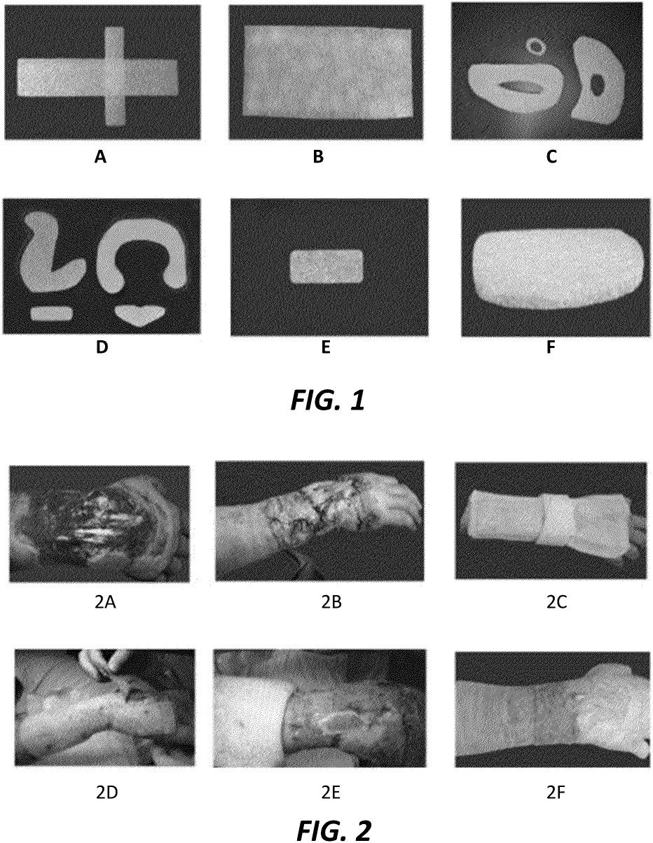

In accordance with the objectives presented by means of the brief description, the present disclosure provides a dressing for treating wounds, according to FIG. 1, which comprises a single layer construction of expanded polymer, applied on a surface in order to exert uniform and homeostatic activity on a wound. The surface where the layer is applied is the surface of a patient’s skin, specifically the surface of the skin that presents a wound, acute or chronic, surgical or resulting from trauma, which needs treatment for correct tissue regeneration and healing. FIG. 1 shows the various exemplary dressing of various formats. Format A is a homeostatic single layer dressing configured to use on upper and lower limbs. Format B is a hemostatic single layer dressing in the form of a plate with a thin layer and indicated for burns and cutaneous injuries. Format C is a homeostatic single layer dressing configured, in some formats, for breast surgery, abdominoplasty or extensive surgeries as brachioplasty and thigh plastic surgery. Format D is a homeostatic single layer dressing configured for face and cervical surgeries. Format E is a homeostatic single layer dressing configured for minor surgeries such as blepharoplasty. Format F is a homeostatic single layer dressing configured for major surgeries, such as skin graft fixation and for cutaneous ulcers.

Said uniform and homeostatic activity is the result of the lightweight of the expanded polymer alone and a stabilizing tension on it, said tension being exerted by the medium used to fix the polymer layer on the skin surface around wound. The means of fixing the polymer single layer to the cutaneous surface may be chosen from the group comprising sutures, adhesive tapes, bandages, elastic bandage wraps, self-adherent wraps or other suitable means available.

Uniform and homeostatic pressure of this dressing allows for microcirculation and regulates the caliber of blood and lymph vessels preventing inappropriate vasodilation in order to stabilize the wound by controlling exudate production and reducing swelling, and the occurrence of rebound bleeds. In the hypothesis of excessive exudate production, the impermeable surface of the lightweight single layer dressing, which is in contact with the skin surface to be treated, moves the secretions to the peripheral portions of the dressing by means of a hydraulic gradient that generates a peristaltic movement without the need to use external forces to drainage.

In the exemplary embodiment of FIG. 1, the construction of the single layer dressing is made with low density polyethylene (LDPE), a polymer formed when ethylene molecules are combined in straight and branched chain with a high degree of short and long chains branching. In some preferred embodiments, the single layer dressing is made of expanded polyethylene foam sheet (EPE Foam Sheet), lightweight with typical density ranging from about 18 to about 25 kg/m³, semi rigid, flexible closed cell foam, harmless, waterproof, dielectric material, near zero moisture absorption, low coefficient of friction, resistant to the growth of microorganisms on its surface, and resistant to many solvents. The thickness of which varies according to the surface where the dressing is applied ranging from about 2 mm to about 10 mm.

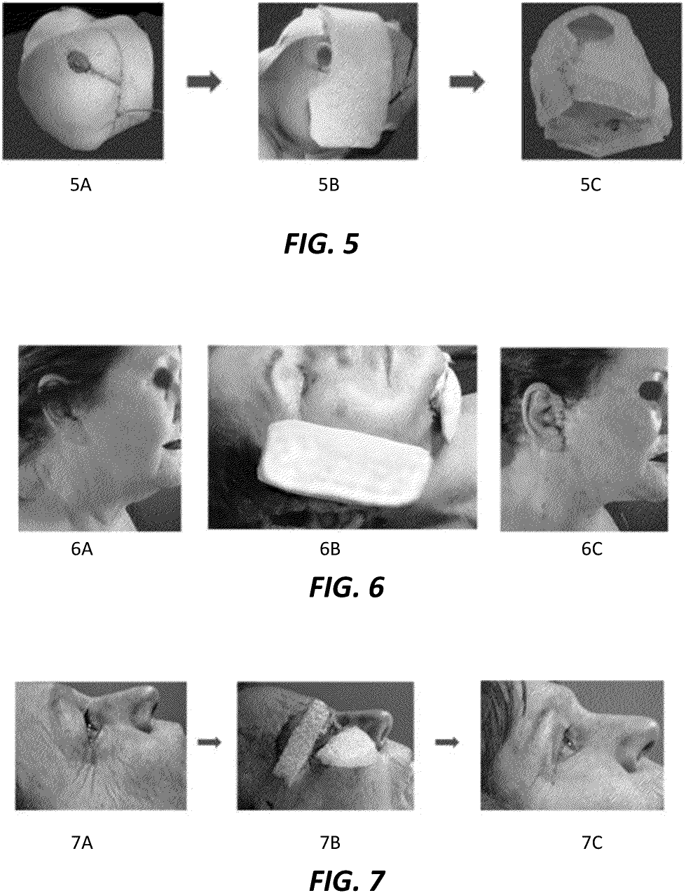

The low elasticity added to the high flexibility of the single layer dressing allows the dressing to assimilate the anatomical shape of the site as demonstrated in FIG. 5, making it difficult to move to other areas, in addition to allowing the construction of spaces for sensors and probes for wound assessment without needing to move the dressing. The possibility of assessing the wound without moving the dressing is an important factor as it reduces the possibility of new lesions in the wound bed, which impairs healing.

In another embodiment of the present disclosure, the surface of the layer constituting the dressing is treated with a sterilizing procedure, which helps in combating the proliferation of pathogens that can impair healing and exacerbate the wound. In some embodiments, the sterilizing procedure is by gamma radiation.

In accordance with the present disclosure, a method of preparing an apparatus for treating wounds and method for treating wounds is described. The a method of preparing an apparatus for treating wounds, which may be carried out using sterile procedure gloves, with care to maintain the integrity of the dressing and ensuring that the dressing remains uncontaminated during the procedure. The method includes a sterilizing treatment of the apparatus for treating wounds. The sterilizing treatment may be accomplished by gamma radiation already known and established by the state of the art for the material that constitutes the layer of the dressing.

The method includes preparing the dressing. The dressing thus prepared is impermeable to water and microorganisms, does not absorb exudate and makes the thermal regulation of the treated area, keeping it as warm as the body. Maintaining a warm temperature at the site favors the metabolism of the growing tissue and is a factor in preventing the proliferation of microorganisms and the formation of biofilm that delay healing and can produce new lesions in an already traumatized tissue. Another advantage of this single-layer dressing is that it acts as a carrier for the release of antimicrobial substances at a lower active dose because it does not absorb these substances, thus reducing cytotoxicity and side effects, which aids in wound recovery.

All the characteristics of the present invention allow the maintenance of the physiological functions of the tissue in recovery, favoring tissue oxygenation by the microcirculation that has its dilation regulated and, consequently, accelerating the metabolism of the cells that constitute the growing tissue. A final advantage of the single layer dressing, also a consequence of the material that forms the single layer and its preparation, consists in the fact that there is no adhesion of the layer to the wound and the skin. Thus, new lesions do not form in the wound bed and the patient stress becomes minimized when changing dressings.

The following examples are provided to illustrate the articles, devices and processes of the present disclosure. The examples are merely illustrative and are not necessarily intended to limit the disclosure to the materials, conditions, or process parameters set forth therein.

With reference to FIG. 2, shown is a dressing of format A, applied to a wound after serve trauma. 2A shows the wound after post-traumatic necrosis. 2B shows the wound of 2A after laminar graft surgery. 2C shows the dressing of Format A applied at the end of the surgery of 2B. 2D and FIG. 2A shows the wound after post-traumatic necrosis. F shows the results five days after the surgery. The removal of the dressing over the grafts without adherence and total graft integration. 2F shows the result after 4 weeks.

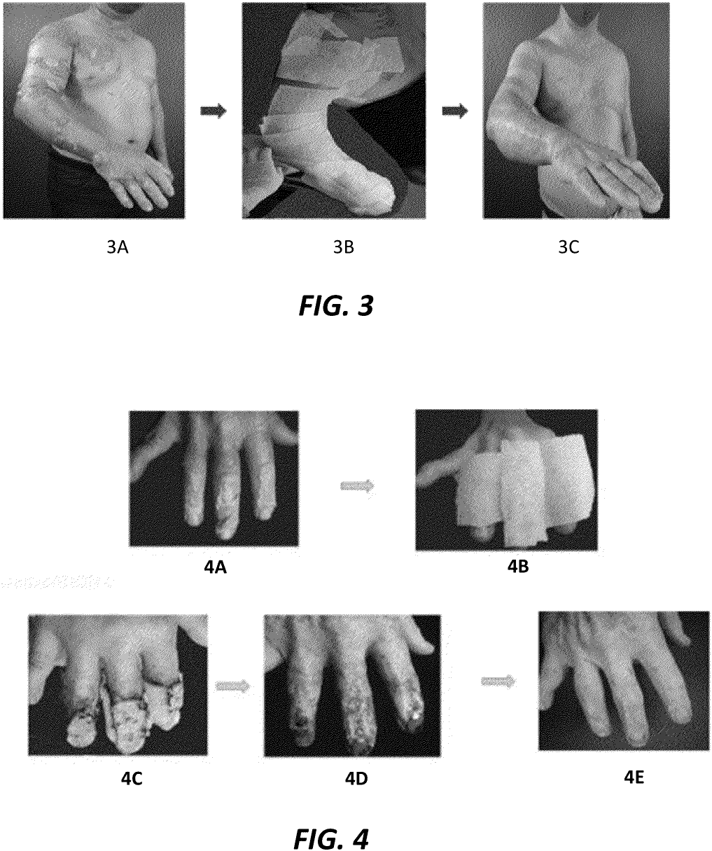

With reference to FIG. 3, shown is the dressing of format B applied to a wound after a petrol burn. 3A shows the wound after a petrol burn. 3B shows application of dressings of format B on the wound. 3C shows the results after one month.

With reference to FIG. 4, shown is the dressing of format B applied to a fourth degree burn involving extensor tendons of the fingers. 4A shows the wound the day of the accident. 4B shows the first dressing of Format B applied to the wound. 4C shows the dressing over the graft. 4D shows the dressing removed after four days. 4E shows the wound two months after grafting.

With reference to FIG. 5, shown is the dressing of format C applied to a mammoplasty. 5A shows an intra operative mammoplasty. 5B shows the dressing of format C applied to the wound at the end of the mammoplasty surgery. 5C shows the dressing frame after removal.

With reference to FIG. 6, shown is the dressing of formats D and F applied after a face lift surgery. 6A shows a side view of a face before a face lift surgery. 6B shows the dressing of formats D and F applied to the wound at the end of the face lift surgery. 6C shows the side view of the face after one week postoperative.

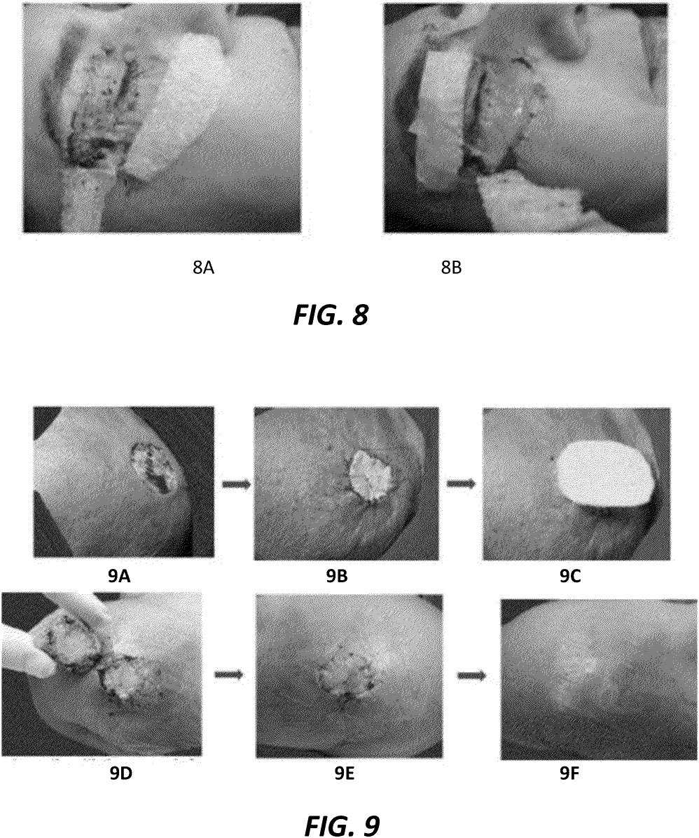

With reference to FIGS. 7 and 8, shown is the dressing of format E applied after a blepharoplasty. 7A shows a side view of a face before the surgery. 7B shows the dressing of format E applied to the wound at the end of the surgery. 7C shows the side view of the face after one month postoperative. In FIG. 8, 8A shows the dressing of format E removed on the fifth day after surgery. The core of the dressings clean and dry (8A). Secretions were displaced around the dressing (8B).

With reference to FIG. 9, shown is the dressing of format F used to fix a skin graft at the end of reconstructive surgery. A tumor was removed presenting the wound of 9A. An intra operative graft is applied at 9B. At 9C a dressing of format F is applied to fix the graft of 9B. The dressing removed four days after post-op is shown at 9D. The dressing was removed without adherence and with a dry inner surface. 9E shows the integrated graft after dressing removal while 9F shows the wound 1 month post-op.

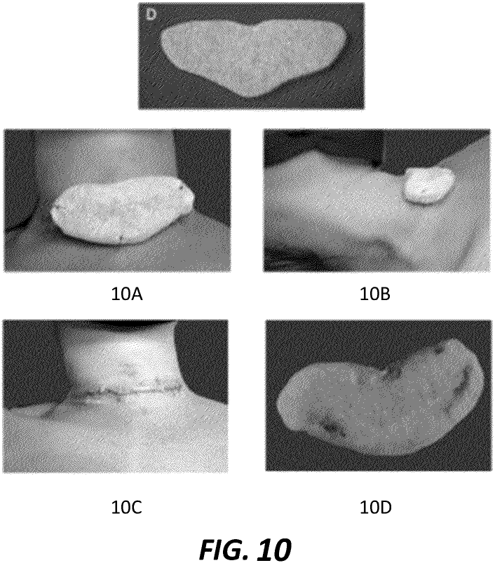

With reference to FIG. 10, shown is the dressing of format D used t at the end of a thrieoidectomy. 10A and 10B show top and side views the dressing of format D. At 10C shown is the wound three days postoperative. After dressing removal, ecchymosis and edema are controlled. At 10D, after removal the core of the dressing of format D is clean and dry. Secretions were displaced around the dressing.

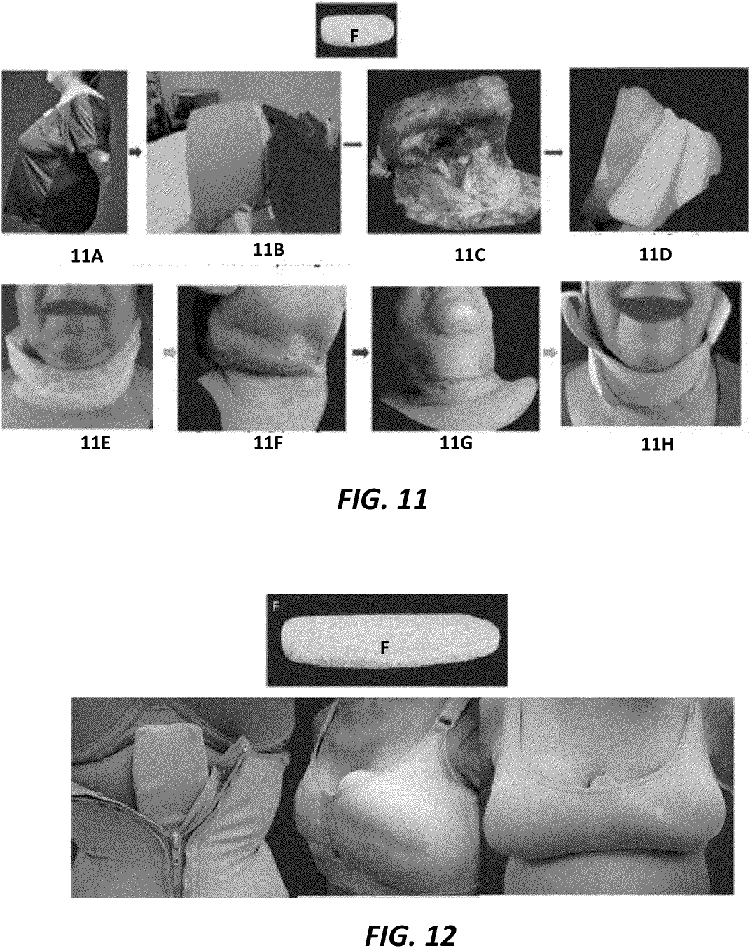

With reference to FIG. 11, shown is the dressing of format F used at the end of head and neck surgery, including neck dissection, a major surgery. At 11A shown is a preoperative view of a patient. 11B shown is an intraoperative view of a patient with a hemostatic stabilization band on the operating table. At 11C is shown an intraoperative view of a neck wound. 11D shows a hemostatic dressing applied at the end of the surgery. 11E and 11F show the patient 7 days postoperative, before and after removal of the hemostatic dressing. 11G and 11H show the patient postoperative after weeks with 11H showing the patient using a hemostatic dressing mask.

With reference to FIG. 12, shown in the dressing of format F (a homeostatic dressing plate) applied under a post-surgical garment on a traumatized body surface. The single layer locally controls edema, seroma, and ecchymosis.

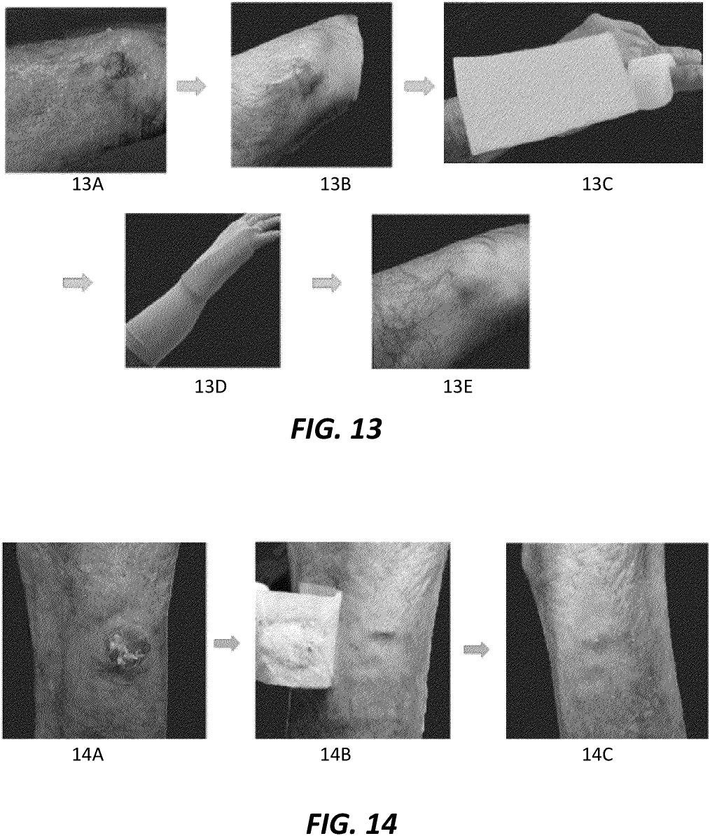

With reference to FIG. 13, shown is a dressing to treat cicatricial hypertrophy. 13A and 13B show two weeks and three months after a burn. 13C includes a dressing modeled before being placed under a garment. At 13D an elastic garment is placed over the dressing of 13C. The elastic garment provides pressure to the dressing and wound. 13E includes the result after three months of pressure.

With reference to FIG. 14, shown is a dressing to treat skin infected ulcer. At 14A shown is a skin ulcer before debridement. At 14B shown is the dressing (such as a dressing of format F) changed every three days, the photo showing the evolution after nine days. At 14C shown is the wound about four weeks after treatment.

While there has been shown and described a preferred embodiment of this invention, it is understood that changes in structure, materials, sizes, and shapes can be made by those skilled in the art without departing from the invention. Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed.

To aid the Patent Office and any readers of this application and anyresulting patent in interpreting the claims appended hereto, applicants do not intend any of the appended claims or claim elements to invoke 35 U.S.C. 112(f) unless the words “means for” or “step for” are explicitly used in the particular claim.

What is claimed is:

1. A homeostatic single-layer dressing consisting of:
a single-layer of expanded polyethylene applied to a wound surface to exert a homeostatic activity on the wound; and
fixing means for attaching the single-layer of expanded polyethylene to the wound surface.

2. The homeostatic single-layer dressing according to claim 1, wherein the homeostatic activity is inherent to the material used, expanded polyethylene foam sheet, and characterized by pressure and temperature gradients.

3. The homeostatic single-layer dressing according to claim 1, wherein the homeostatic activity is a pressure exerted by a weight of the single-layer of expanded polyethylene on the wound surface sufficient to control microcirculation.

4. The homeostatic single-layer dressing according to claim 3, wherein the homeostatic activity on the wound surface generates a hydraulic gradient between the wound surface and a skin around the wound surface so that wound secretions are swept to an edge line of the single-layer of expanded polyethylene.

5. The homeostatic single-layer dressing according to claim 1, wherein a material of the single-layer of expanded polyethylene is isothermal and configured to maintain a body temperature in a wound environment without drying the wound surface out while maintaining physiological moisture.

6. The homeostatic single-layer dressing according to claim 1, wherein the single-layer of expanded polyethylene has a thickness ranging from about 2 mm to about 10 mm.

7. The homeostatic single-layer dressing according to claim 1, wherein a shape and a dimension of the single-layer of expanded polyethylene varies according to the wound surface where the said single-layer of expanded polyethylene is applied.

8. The homeostatic single-layer dressing according to claim 1, wherein the fixing means comprises one of sutures, adhesive tapes, bandages, and elastic cotton fabrics.

9. The homeostatic single layer dressing according to claim 8, wherein the single-layer of expanded polyethylene supports sutures, the sutures being supported by the single layer of expanded polyethylene such that the sutures do not tear.

10. The homeostatic single-layer dressing according to claim 1, wherein the single-layer of expanded polyethylene is sterilized according to a sterilizing procedure.

11. The homeostatic single-layer dressing, according to claim 10, wherein the sterilizing procedure is gamma radiation.

12. The homeostatic single-layer dressing according to claim 1, wherein the single-layer of expanded polyethylene is a flexible closed cell foam.

13. A method for treating a wound consisting of:
providing a single-layer of expanded polyethylene;
applying the single-layer of expanded polyethylene to the wound, wherein the applying comprises fixing the single-layer of expanded polyethylene by fixing means to the wound; and
exerting a homeostatic activity on the wound by an application of the single-layer of expanded polyethylene to the wound.

14. The method according to claim 13 further comprising:
controlling a microcirculation of the wound by a weight of the single-layer of expanded polyethylene exerting on the wound.

15. The method according to claim 13, wherein the homeostatic activity on the wound generates a hydraulic gradient between the wound and a skin around the wound, and wherein wound secretions are swept to an edge line of the single-layer of expanded polyethylene.

16. The method according to claim 13, wherein the fixing means are connected to the single-layer of expanded polyethylene and comprises one of sutures, adhesive tapes, bandages, and elastic cotton fabrics and is applied to a skin to apply the single-layer of expanded polyethylene to the wound.

17. The method according to claim 13 further comprising:
sterilizing at least one surface of the single-layer of expanded polyethylene.

18. The method according to claim 17, wherein sterilizing at least one surface of the single-layer of expanded polyethylene comprises application of gamma radiation.

19. The homeostatic single-layer dressing according to claim 1, wherein the single-layer of expanded polyethylene is applied to the wound surface of a wound to directly contact the wound.
